(19) United States
(12) Patent Application Publication
(10) Pub. No.: US 2016/0153002 A1
(43) Pub. Date: Jun. 2, 2016

(54) METHOD FOR CELL MEMBRANE PERMEATION FOR COMPOUND

(71) Applicant: HitGen LTD., Chengdu (CN)

(72) Inventors: Jin Li, Chengdu (CN); Benyanzi Yang, Chengdu (CN); Dengfeng Dou, Chengdu (CN); Xiaohu Ge, Chengdu (CN); Hongmei Song, Chengdu (CN); Jinqiao Wan, Chengdu (CN); Yan Zhang, Chengdu (CN); Xiao Hu, Chengdu (CN); Xing Wang, Chengdu (CN); Jingchao Feng, Chengdu (CN); Guoqing Zhong, Chengdu (CN)

(21) Appl. No.: 14/948,201

(22) Filed: Nov. 20, 2015

Related U.S. Application Data
(63) Continuation-in-part of application No. PCT/CN2014/077971, filed on May 21, 2014.

(30) Foreign Application Priority Data
May 21, 2013 (CN) 201310190207.5

(51) Int. Cl. C12N 15/87; C12N 15/113

(52) U.S. Cl. CPC C12N 15/87; C12N 15/113; C12N 2310/14; C12N 2310/351

(57) ABSTRACT

The present invention discloses a cell-penetrating method for compounds, comprising the following steps of: (1) preparing raw materials, i.e., the compounds and DNA or RNA; (2) linking: linking the compounds to the DNA or RNA to obtain a molecular conjugate; and (3) transferring: transferring the molecular conjugate obtained in the step (2) into cells by a gene transfer method. The present invention further discloses a structure of a molecular conjugate for transmembrane transfer and a method for synthesizing the molecular conjugate. The method of the present invention effectively solves a problem of low membrane permeability of compounds, so that the compounds enter the cell to act on targets thereof, thus to provide a novel drug-delivery way. The method of the present invention may be used for clinical treatment by drugs with low membrane permeability. This method significantly increases the quantity of potential drugs, and the clinical application of many drugs which are eliminated due to their low membrane permeability becomes possible. The method of the present invention may be used for capturing unknown targets of drugs in cells and conducting researches on the target mechanism. This method significantly shortens the course of research and development of drugs and has excellent application prospect.

### CROSS-REFERENCE TO RELATED APPLICATIONS

This application is a continuation-in-part of International Patent Application No. PCT/CN2014/077971 with an international filing date of May 21, 2014, designating the United States, now pending, and further claims priority benefits to Chinese Patent Application No. 201310190207.5 filed May 21, 2013. The contents of all of the aforementioned applications, including any intervening amendments thereto, are incorporated herein by reference.

### SEQUENCE LISTING

This application contains, as a separate part of the disclosure, a Sequence Listing in computer-readable form (filename: wk15_083_ST25.txt; created: Nov. 19, 2015; 638 bytes—ASCII text file) which is incorporated by reference in its entirety.

### TECHNICAL FIELD

The present invention relates to a cell-penetrating method for compounds.

### BACKGROUND OF THE PRESENT INVENTION

For drug targets in some cells, small-molecule drugs need to penetrate through the cell membrane to be bound to related target sites, thus to show bioactivity. Due to structural features of the cell membrane, small molecules which have a large molecular weight or a large molecular polarity or which are likely to be charged are difficult to penetrate through the cell membrane to the biological target sites, and thus unable to show related activity. Some small molecules, showing excellent activity in bioassays on the molecular level, cannot show bioactivity at the cellular level. An important factor is that the small molecules themselves cannot penetrate through the cell membrane. How to improve the membrane permeability of small molecules is the key to solve this problem.

An existing method to improve the membrane permeability of small-molecule drugs is to modify the small molecules directly, for example, to turn the small molecules into prodrugs, or to carry the small molecules into cells by using other materials (for example, nano materials and cell-penetrating peptides (CPPs)) as carriers. However, modification to the small molecules themselves has a high risk that it may be impossible to maintain the activity of the small molecules themselves. [Journal of Medicinal Chemistry, 2002, 45, 4443-4459] Moreover, conventional carriers have the following deficiencies such as complicated operation, high cost, large difference in transfer efficiency for different drug molecules, low stability of the complexes, or presence of cytotoxicity of the transfer materials themselves. [Drug Discov Today Technol 49-55] Therefore, seeking for a cell-penetrating method for small-molecule compounds, which is easy in operation, high in transfer efficiency, can maintain the activity of small-molecule compounds to the maximum extent and is safe and non-poisonous, is of great significance for earlier research and clinical treatment of drugs.

### SUMMARY OF THE PRESENT INVENTION

In order to solve the aforementioned problem, the present invention provides a cell-penetrating method for compounds, as well as a molecular conjugate for transmembrane transfer as shown in Structural Formula 1 and a method for synthesizing the molecular conjugate.

The cell-penetrating method for compounds of the resent invention includes the following steps of:

(1) Preparing raw materials, i.e., the compounds and DNA or RNA;

(2) Linking: linking the compounds to the DNA or RNA to obtain a molecular conjugate as shown in FIG. 1; and

(3) Transferring: transferring the molecular conjugate obtained in the step (2) into cells by a gene transfer method.

In the step (1), the compounds are small-molecule compounds or polypeptides having a molecular weight of 100 Da to 4000 Da.

In the step (1), the DNA or RNA is any sequence having a length not less than five bases or base pairs.

In one specific implementation, the DNA or RNA linked to the compounds may be: polyA of 5 bp, polyA of 19 bp, polyA of 38 bp, a single-stranded random sequence of 19 bp or a double-stranded random sequence of 19 bp.

In the step (1), the DNA or RNA is single-stranded or double-stranded. An end-chain or in-chain covalent bond of the DNA or RNA is bound with zero or a plurality of tags. The tag is fluorescent or isotopic.

In the step (2), the compounds are linked to the DNA or RNA by a linker. The linker is formed by linking any saturated and non-saturated covalent groups capable of modifying the compounds and the DNA/RNA.

In the step (3), the gene transfer method refers to a cationic liposome transfection method, a calcium phosphate transfection method, a nanoparticles transfection method or an electroporation transfection method and other technical methods capable of transferring nucleic acid into cells.

The “gene transfer” refers to a process of transferring nucleic acid into cells physically, chemically or biologically.

A molecular conjugate, having a structural formula as follows:

X-linker-DNA/RNA Formula 1

where X refers to compounds with low membrane permeability, and linker is a linker between X and DNA or RNA.

The compounds are small-molecule compounds or polypeptides having a molecular weight of 100 Da to 4000 Da.

The DNA or RNA is any sequence having a length not less than five bases or base pairs.

The DNA or RNA is single-stranded or double-stranded.

An end-chain or in-chain covalent bond of the DNA or RNA is bound with zero or a plurality of tags.

The tag is fluorescent or isotopic.

The linker is formed by linking any saturated and non-saturated covalent groups capable of modifying the compounds and the DNA/RNA.

In one specific implementation, the structural formula of the molecular conjugate prepared in the present invention may be any one of the following four structural formulas: in FIG. 1a

With the method of the present invention, the compounds having low membrane permeability are linked to the DNA or RNA to obtain a molecular conjugate which can perform transmembrane transfer and then, by the gene transfer method, for example, the cationic liposome transfection method, the calcium phosphate transfection method, the nanoparticles transfection method, the electroporation transfection method and other technical methods capable of transferring nucleic acid into cells, the molecular conjugate is transferred into cells, so that the compounds having low membrane permeability may act inside the cell. This method makes the application of drugs having low membrane permeability in clinic treatment become possible, and has excellent application prospect.

Apparently, according to the content of the present invention, various modifications, replacements and alterations in other forms may be made in accordance with common technical knowledge and conventional methods of the art, without departing from the basic technical concept of the present invention.

The content of the present invention will be further described in detail by specific implementations in the form of embodiments. However, it should not be interpreted as limiting the scope of the subject of the present invention only to the following embodiments. Techniques realized based on the content of the present invention shall all fall into the scope of the present invention.

### BRIEF DESCRIPTION OF THE DRAWINGS

FIG. 1 is a structural diagram of a molecular conjugate for transmembrane transfer according to the present invention;

FIG. 1a shows the possible structural formula of the molecular conjugate prepared in the present invention

FIG. 2-1 is an exemplary synthesis route of a molecular conjugate 1;

FIG. 2-2 is an exemplary synthesis route of a molecular conjugate 2;

FIG. 2-3 is an exemplary synthesis route of a molecular conjugate 3;

FIG. 2-4 is an exemplary synthesis route of a molecular conjugate 4;

FIG. 3-1 is a ¹H NMR curve of a compound 1-3;

FIG. 3-2 is a ¹H NMR curve of a compound 1-5;

FIG. 4-1 is HPLC purity analysis of the molecular conjugate 1;

FIG. 4-2 is mass-spectrometric analysis of the molecular conjugate 1;

FIG. 5 shows positioning, by laser confocal microscopy, in cell-penetrating experiments, of molecular conjugates having single-stranded or double-stranded DNA or RNA of different length, different compounds and different linkers (those in blue are cell nucleus, and those in green are FITC-tagged single-stranded or double-stranded DNA or RNA);

FIG. 6: A) shows positioning, by laser confocal microscopy, in cell-penetrating experiments, of FITC-separately-tagged 4-bromo-3-oxo-ethyl cyclopropanecarboxylate-5-(3-((1-phenyldiethyl carbamoylpiperidine)-4-methyl)phenyl)thiophene-2-methyl formate (compound 1-1); and B) shows positioning, by laser confocal microscopy, in cell-penetrating experiments, of the molecular conjugate 1 (those in blue are cell nucleus, and those in green are FITC-tagged molecular conjugates 1);

FIG. 7: A) shows the total number of cells in transfer experiments, observed by a phase contrast microscope; B) shows the total number of cells into which the molecular conjugates 1 are successively transferred, observed by a fluorescent microscope; and C) shows the statistics of the transfer efficiency of the molecular conjugate 1; and

FIG. 8 shows the influence, on the phosphorylation of cells, of transferring compounds having an effect of the PTP1B inhibitor into the cells in the form of molecular conjugates. in which, 1: X-tremeGENEsiRNA reagent; 2: molecular conjugate 1 (5 nM); 3: molecular conjugate 1 plus X-tremeGENEsiRNA reagent (5 nM); and 4: molecular conjugate 1 plus X-tremeGENEsiRNA reagent (15 nM).

### DETAILED DESCRIPTION OF THE PRESENT INVENTION

### Embodiment 1

### Preparation of a Molecular Conjugate for Transmembrane Transfer by the Method of the Present Invention

1. Experimental Materials and Reagents

The molecular conjugates 1 were synthesized by our company according to the method as described in the reference document (D. P. Wilson et al, J. Med. Chem. 2007, 50, 4681-4698); polyA (5′-(CH2)12-A19-3′-FITC) modified by 5′-amino and 3′-fluorescein was purchased from Invitrogen Trading Shanghai Co., Ltd; and the other reagents used for chemical synthesis were purchased from Aldrich or TCI.

2. Synthesis Method

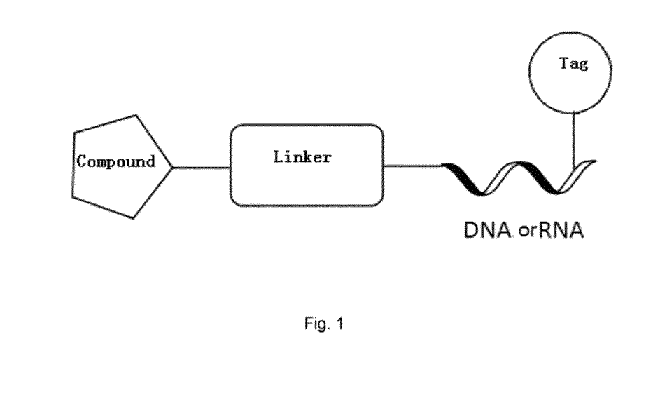

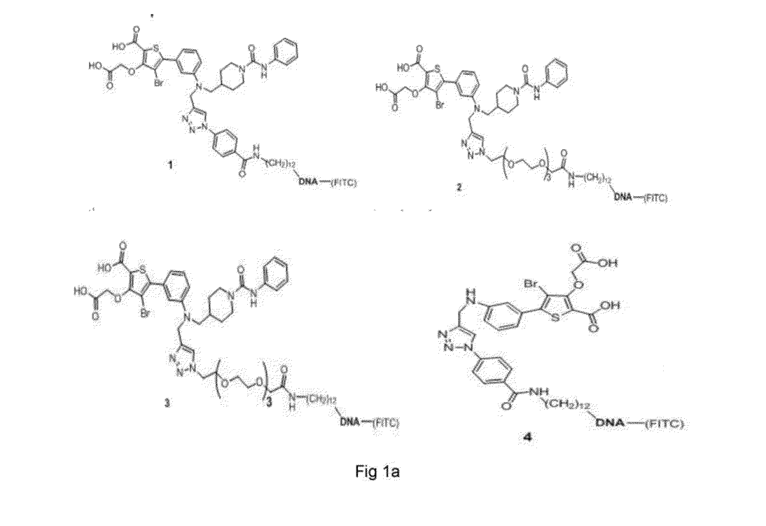

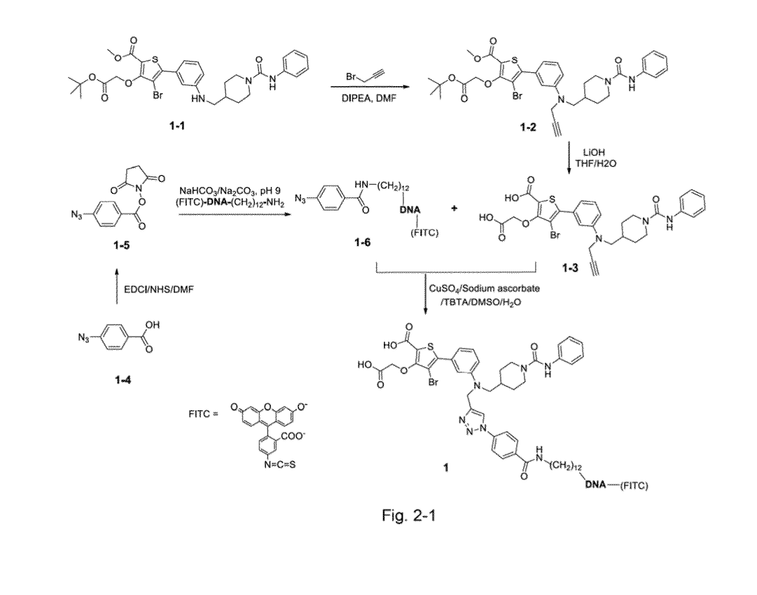

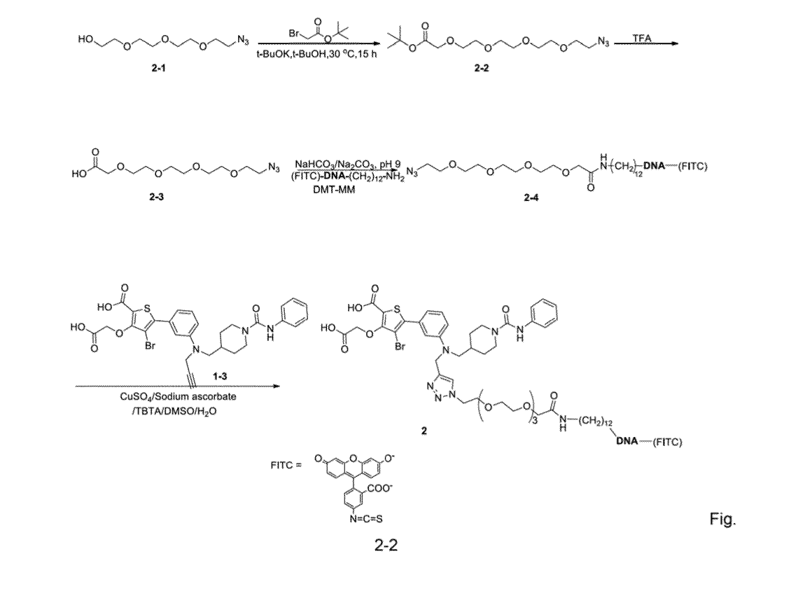

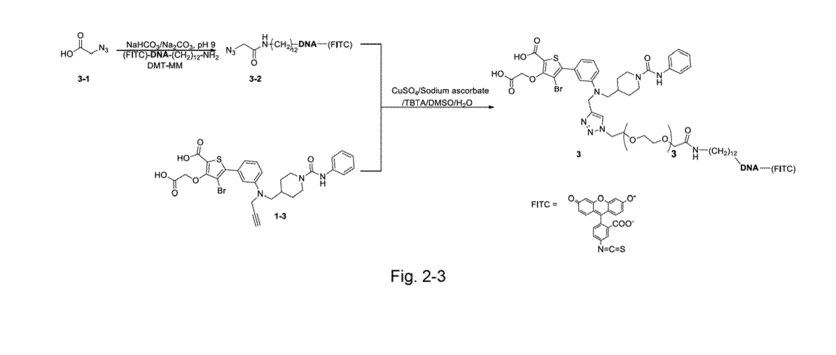

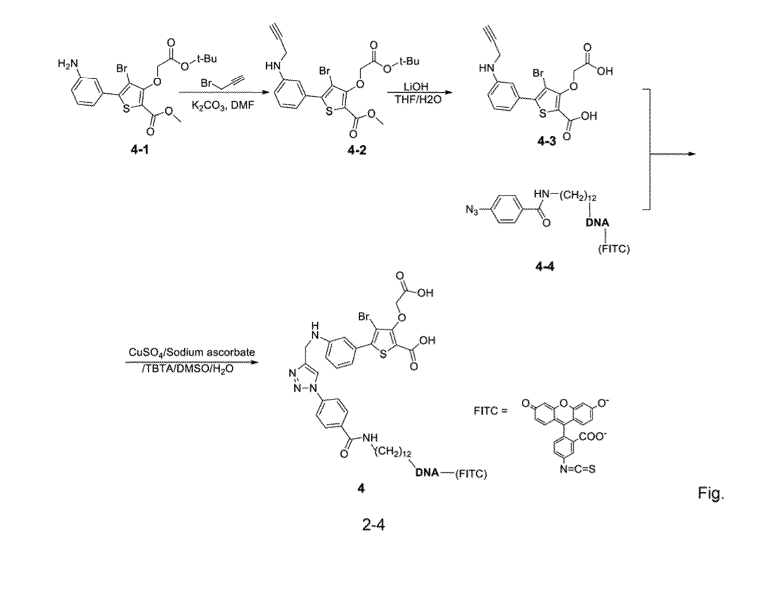

(1) Synthesis Route of the Molecular Conjugate 1 (as Shown in FIG. 2-1).

### Synthesis of compound 1-2: 4-bromo-3-oxo-tert-butyl acetate-5-(3-(((1-phenyl carbamoylpiperidine)-4-methyl)-N-propargyl amine)phenyl)thiophene-2-methyl formate

4-bromo-3-oxo-tert-butyl acetate-5-(3-(((1-phenyl carbamoylpiperidine)-4-methyl)-phenyl)thiophene-2-methyl formate (compound 1-1) (250 mg, 0.4 mmol), propargyl bromide (70 mg, 0.5 mmol) and N,N-diisopropylethylamine (1.5 mL) were dissolved into 20 mL of N,N-dimethylformamide, stirred for 5 h at 90° C., cooled to room temperature, and distilled under reduced pressure to obtain a crude product; and the crude product was separated by column chromatography to obtain 4-bromo-3-oxo-tert-butyl acetate-5-(3-(((1-phenyl carbamoylpiperidine)-4-methyl)-N-propargyl amine)phenyl)thiophene-2-methyl formate (compound 1-2) (white solid, 130 mg, with a yield of 49%). MS m/z (ESI): 668,670(M+H)⁺; 690,692 (M+Na)⁺.

### Synthesis of compound 1-3: 4-bromo-3-oxoacetic acid-5-(3-(((1-phenyl carbamoylpiperidine)-4-methyl)-N-propargyl amine)phenyl)thiophene-2-formic acid

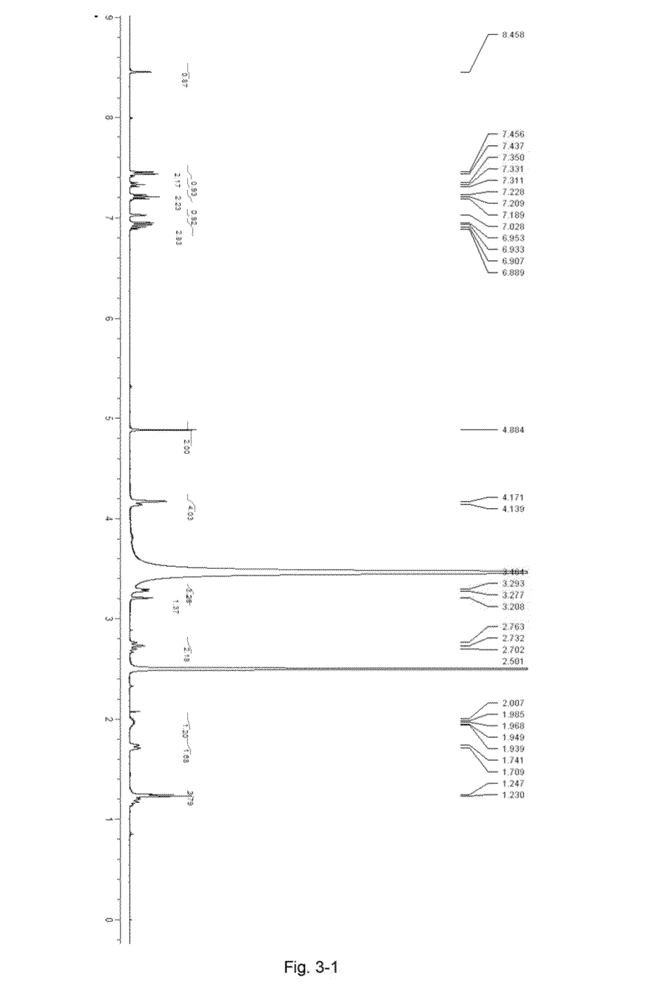

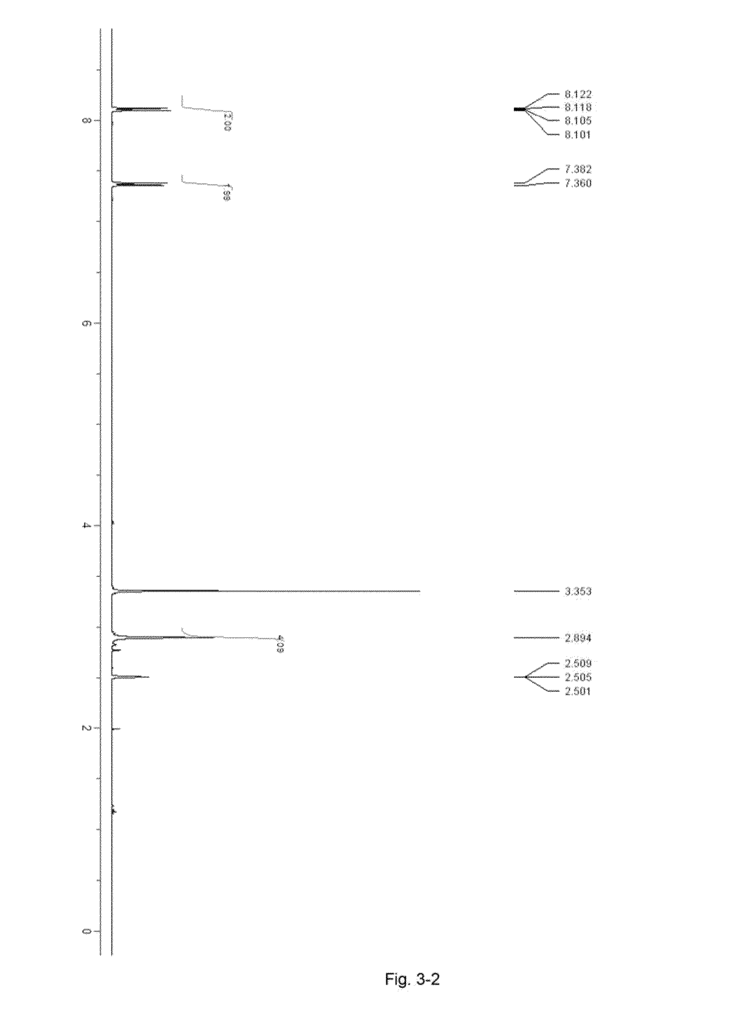

Lithium hydroxide (200 mg, 2.38 mmol) was added to 4-bromo-3-oxo-tert-butyl acetate-5-(3-(((1-phenyl carbamoylpiperidine)-4-methyl)-N-propargyl amine)phenyl)thiophene-2-methyl formate (compound 1-2) (100 mg, 0.15 mmol) in 5 mL of tetrahydrofuran and 5 mL of aqueous solution, and stirred overnight at room temperature. 2N hydrochloric acid was added to the reaction solution; and the reaction solution was acidified until the pH became 2, and then concentrated to obtain a crude product. The crude product was treated by HPLC to obtain 4-bromo-3-oxoacetic acid-5-(3-(((1-phenyl carbamoylpiperidine)-4-methyl)-N-propargyl amine)phenyl)thiophene-2-formic acid (compound 1-3) (white solid, 40 mg, with a yield of 42%). MS m/z (ESI): 626,628 (M+H)⁺; ¹H NMR (CDCl3): δ8.45 (s, 1H), 7.43 (m, 2H), 7.33 (t, J=7.6 Hz, 1H), 7.21 (m, 2H), 7.03 (s, 1H), 6.92 (m, 3H), 4.88 (s, 2H), 4.15 (m, 4H), 3.28 (m, 2H), 3.20 (m, 1H), 2.70 (m, 2H), 1.82 (m, 1H), 1.73 (m, 2H), 1.24 (m, 3H). (See FIG. 3-1 for ¹H NMR)

### Synthesis of compound 1-5: 4-azidobenzoate succinimide ester

In ice bath, 1-ethyl-3-(3-dimethylamine propyl) carbodiimide hydrochloride (EDCl, 570 mg, 3.7 mmol) was added to 10 mL of N,N-dimethylformamide containing 4-azidobenzoic acid (compound 1-4) (500 mg, 3.06 mmol), and then N-hydroxysuccinimide (440 mg, 3.7 mmol) was added thereto. The reaction lasted for 1 h away from light and under protection of nitrogen; and then, the reaction solution was heated to room temperature, and stirred overnight away from light. N,N-dimethylformamide was removed by distillation under reduced pressure; and then, the residues were dissolved in ethyl acetate and washed with water for three times; and finally, the organic phase was dried by anhydrous sodium sulfate, filtered and concentrated to obtain a crude product. The crude product was separated by column chromatography to obtain the product 4-azidobenzoate succinimide ester (compound 1-5) (white solid, 780 mg, with a yield of 97.5%). ¹H NMR (DMSO-d₆): δ8.11 (d, J=8.4 Hz, 2H), 7.37 (d, J=8.4 Hz, 2H), 7.37 (s, 4H). (See FIG. 3-2 for ¹H NMR)

### Synthesis of compound 1-6: 4-azidobenzamide 12-alkyl 19 polyA fluorescein

A mixture of polyA (5′-(CH₂)₁₂-A₁₉-3′-FITC) (50 nmol) modified by 5′-amino and 3′-fluorescein, the 4-azidobenzoate succinimide ester (compound 1-5) (5 μmol, 100 eq.) in 500 μL of 0.5 M sodium carbonate/sodium bicarbonate buffer solution (pH 9), and 500 μL of dimethylsulfoxide was shaken overnight in a low speed at room temperature. Then, the reaction system was directly separated by reverse HPLC column chromatography, and lyophilized to obtain 4-azidobenzamide 12-alkyl 19 polyA fluorescein (compound 1-6) (light yellow solid, with a yield of over 90%).

### Synthesis of molecular conjugate 1: 4-bromo-3-oxoacetic acid-5-(3-(((1-(4-fluorescein 19 polyA) 12-alkyl acetamidophenyl)-1H-1,2,3-triazole-4-methylene)(1-phenylcarbamoylpiperidine)-4-methyl)amino)phenyl)thiophene-2-formic acid

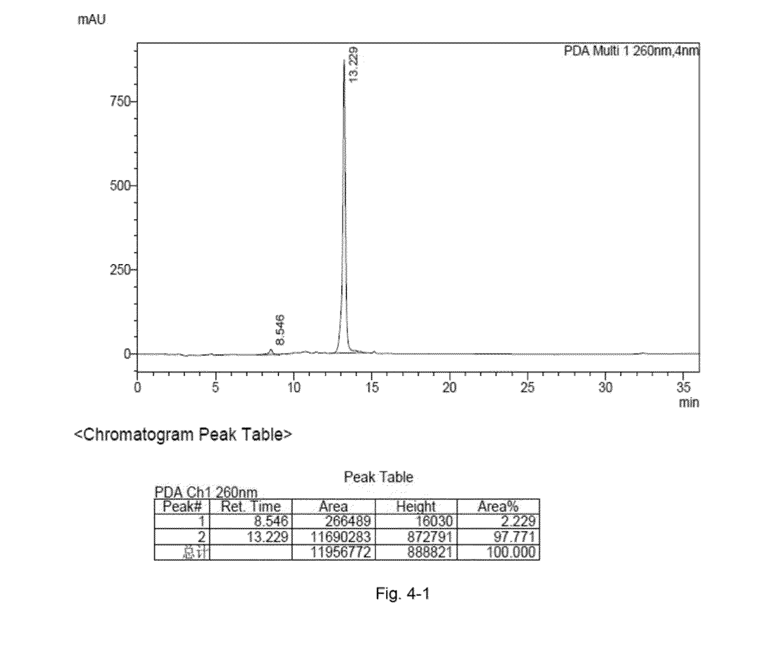

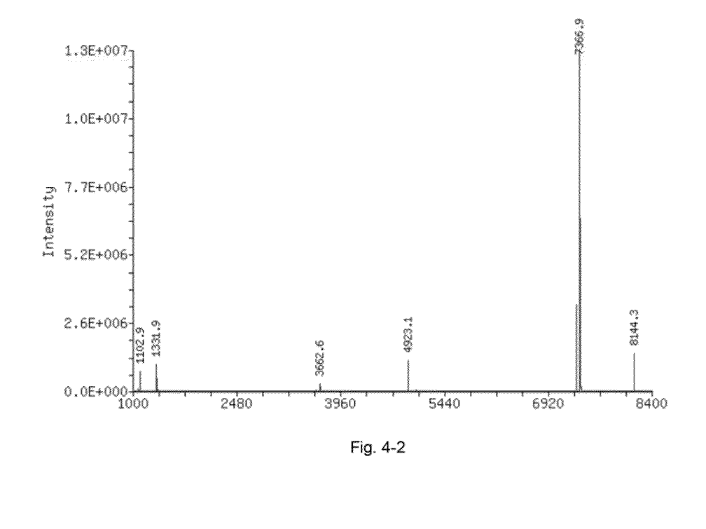

30 μL of solution A (copper sulfate and tris[(1-benzyl-1H-1,2,3-triazol-4-yl)methyl]amine were dissolved at a mole ratio of 1:2 into a solution composed of water, dimethylsulfoxide and tert-butanol at a volume ratio of 4:3:1, with a concentration of 10 mM) was added to solution B (4-azidobenzamide 12-alkyl 19 polyA fluorescein (compound 1-6) (15 nmol) in 200 μL of aqueous solution and 4-bromo-3-oxoacetic acid-5-(3-(((1-phenyl carbamoylpiperidine)-4-methyl)-N-propargyl amine)phenyl)thiophene-2-formic acid (compound 1-3) (960 nmol)) in 50 μL of DMSO solution, and vortex-centrifuged; and subsequently, 60 μL of newly-prepared sodium ascorbate (600 nmol) in aqueous solution was added to the reaction system, and then shaken overnight in a low speed at room temperature. Then, the reaction solution was directly separated by reverse HPLC column chromatography and purified to obtain the product 4-bromo-3-oxoacetic acid-5-(3-(((1-(4-fluorescein 19 polyA) 12-alkyl acetamidophenyl)-1H-1,2,3-triazole-4-methylene)(1-phenylcarbamoylpiperidine)-4-methyl)amino)phenyl)thiophene-2-formic acid (molecular conjugate 1) (light yellow solid, with a yield of 80%). (The HPLC purity analysis of the molecular conjugate 1 is as shown in FIG. 4-1, and the mass-spectrometric analysis of the molecular conjugate 1 is as shown in FIG. 4-2)

(2) Synthesis Route of the Molecular Conjugate 2 (as Shown in FIG. 2-2).

### Synthesis of compound 2-2 14-azido-3,6,9,12-tetraoxatetradecyl-1-tert-butyl carboxyl

Potassium tert-butylate (333 mg, 3 mmol) was added to 15 mL of tert-butanol solution (compound 2-1) (372 mg, 2 mmol), and stirred for 15 min at 30° C. Then, tert-butyl bromoacetate (780 mg, 4 mmol) was added to the system, and stirred overnight at 30° C. The system is distilled under reduced pressure to obtain a crude product. The crude product was dissolved into 30 mL of dichloromethane, and washed with water for three times and with saturated salt water for three times successively; and the organic phase was dried by anhydrous sodium sulfate, filtered and concentrated to obtain a compound 2-2 (Clear oily liquid, 466 mg, with a yield of 70%). MS m/z (ESI): 250(M-tBu-N₂+H)⁺; 278(M-tBu+H)⁺.

### Synthesis of compound 2-3 14-azido-3,6,9,12-tetraoxatetradecyl-1-carboxyl

Trifluoroacetic acid (1 mL) was added to the compound 2-2 (466 mg, 1.4 mmol) in 5 mL of dichloromethane solution, and stirred for 2 h at room temperature. The solution is concentrated to obtain a crude product compound 2-3 (clear oily liquid, 370 mg, with a yield of 95%). MS m/z (ESI): 250(M-N₂+H)⁺; 278(M+H)⁺.

### Synthesis of compound 2-4 14-azido-3,6,9,12-tetraoxatetradecyl-1-formyl-n-dodecyl 19 polyA fluorescein

A mixture of polyA (5′-(CH₂)₁₂-A₁₉-3′-FITC) (80 nmol) modified by 5′-amino and 3′-fluorescein, the compound 2-3 (1.6 μmol, 200 eq.), 4-(4,6-dimethoxy triazin-2yl)-4-methyl morpholine hydrochloride (DMT-MM, 1.6 μmol, 200 eq.) in 80 μL of 0.5 M sodium carbonate/sodium bicarbonate buffer solution (pH 9), 160 μL of deionized water and 160 μL of dimethylsulfoxide was shaken overnight in a low speed at room temperature. Then, the reaction system was directly separated by reverse HPLC column chromatography and lyophilized to obtain the compound 2-4 (white solid). MS m/z (TOF): 6896

### Synthesis of the Molecular Conjugate 2

60 μL of solution A (copper sulfate and tris[(1-benzyl-1H-1,2,3-triazol-4-yl)methyl]amine were dissolved at a mole ratio of 1:2 into a solution composed of water, dimethylsulfoxide and tert-butanol at a volume ratio of 4:3:1, with a concentration of 10 mM) was added to solution B (compound 2-4 (50 nmol) in 400 μL of aqueous solution and 4-bromo-3-oxoacetic acid-5-(3-(((1-phenyl carbamoylpiperidine)-4-methyl)-N-propargyl amine)phenyl)thiophene-2-formic acid (compound 1-3) (3 umol)) in 100 μL of DMSO solution, and vortex-centrifuged; and subsequently, 120 μL of newly-prepared sodium ascorbate (1200 nmol) in aqueous solution was added to the reaction system, and shaken overnight in a low speed at room temperature. Then, the reaction solution was directly separated by reverse HPLC column chromatography and purified to obtain the molecular conjugate 2 (light yellow solid). MS m/z (TOF): 7521

(3) Synthesis Route of the Molecular Conjugate 3 (as Shown in FIG. 2-3).

### Synthesis of Compound 3-2

A mixture of polyA (5′-(CH₂)₁₂-A₁₉-3′-FITC) (80 nmol) modified by 5′-amino and 3′-fluorescein, azidoacetic acid (the compound 3-1) (1.6 μmol, 200 eq.), 4-(4,6-dimethoxy triazin-2-yl)-4-methyl morpholine hydrochloride (DMT-MM, 1.6 μmol, 200 eq.) in 80 μL of 0.5 M sodium carbonate/sodium bicarbonate buffer solution (pH 9), 160 μL of deionized water and 160 μL of dimethylsulfoxide was shaken overnight in a low speed at room temperature. Then, the reaction system was directly separated by reverse HPLC column chromatography and lyophilized to obtain the compound 3-2 (white solid). MS m/z (TOF): 6720

### Synthesis of the Molecular Conjugate 3

60 μL of solution A (copper sulfate and tris[(1-benzyl-1H-1,2,3-triazol-4-yl)methyl]amine were dissolved at a mole ratio of 1:2 into a solution composed of water, dimethylsulfoxide and tert-butanol at a volume ratio of 4:3:1, with a concentration of 10 mM) was added to solution B (compound 3-4 (50 nmol) in 400 μL of aqueous solution and 4-bromo-3-oxoacetic acid-5-(3-(((1-phenyl carbamoylpiperidine)-4-methyl)-N-propargyl amine)phenyl)thiophene-2-formic acid (compound 1-3) (3 umol)) in 100 μL of DMSO solution, and vortex-centrifuged; and subsequently, 120 μL of newly-prepared sodium ascorbate (1200 nmol) in aqueous solution was added to the reaction system, and then shaken overnight in a low speed at room temperature. Then, the reaction solution was directly separated by reverse HPLC column chromatography and purified to obtain the molecular conjugate 3 (light yellow solid). MS m/z (TOF): 7345

(4) Synthesis Route of the Molecular Conjugate 4 (as Shown in FIG. 2-4).

### Synthesis of Compound 4-2

The compound 4-1 (441 mg, 1 mmol), propargyl bromide (95 mg, 0.8 mmol) and potassium carbonate (138 mg, 1 mmol) were dissolved into 20 mL of N,N-dimethylformamide, and stirred overnight at room temperature. The system is distilled under reduced pressure to obtain a crude product. The crude product was dissolved into 50 mL of dichloromethane, and washed with water for three times and with saturated salt water for three times successively; the organic phase was dried by anhydrous sodium sulfate, filtered and concentrated to obtain a compound 4-2 (yellow solid, 287 mg, with a yield of 60%). MS m/z (ESI): 424(M-tBu+H)⁺; 480 (M+H)⁺.

### Synthesis of Compound 4-3

The compound 4-2 (87 mg, 0.6 mmol) and lithium hydroxide monohydrate (126 mg, 3 mmol) were dissolved into 5 mL of methanol and 5 mL of water, and reflux-stirred overnight. Ethanol was removed by distillation. The solution was diluted with 20 mL of water and acidified with 1N HCl until the pH became 2.0, and lyophilized to obtain a crude product; and the crude product was directly subject to reverse high-phase liquid-phase separation to obtain the compound 4-3 (yellow solid, 216 mg, with a yield of 80%). MS m/z (ESI): 410(M+H)⁺.

### Synthesis of the Molecular Conjugate 4

60 μL solution A (copper sulfate and tris[(1-benzyl-1H-1,2,3-triazol-4-yl)methyl]amine were dissolved at a mole ratio of 1:2 into a solution composed of water, dimethylsulfoxide and tert-butanol at a volume ratio of 4:3:1, with a concentration of 10 mM) was added to solution B (the compound 4-4 (50 nmol) in 400 μL of aqueous solution and the compound 4-4 (50 nmol) in 100 μL of DMSO solution), and vortex-centrifuged; and subsequently, 120 μL of newly-prepared sodium ascorbate (1200 nmol) in aqueous solution was added to the reaction system, and then shaken overnight in a low speed at room temperature. Then, the reaction solution was directly separated by reverse HPLC column chromatography and purified to obtain the molecular conjugate 4 (light yellow solid). MS m/z (TOF): 7191

### Embodiment 2

Evaluation on transmembrane transfer efficiency of the single-stranded or double-stranded DNA or RNA

1. Experimental Materials and Reagents

The HepG2 cell strains were purchased from Shanghai Institutes for Bioscience Chinese Academy of Sciences; the RPMI-1640 culture medium was purchased from Hyclone Shanghai; the fetal bovine serum was purchased from Tianjin Hao Yang Biological Products Co., Ltd.; the trypsin and Opti-MEM were purchased from Invitrogen Shanghai; the X-tremeGENEsiRNA transfection reagent was purchased from Roche China; and the cell culture dishes and other consumables were all purchased from Corning China.

polyA of 5 bp: 5′-NH₂—(CH₂)₁₂—PO₄-A₅-3′-FITC,

polyA of 19 bp: 5′-NH₂—(CH₂)₁₂—PO₄-A₁₉-3′-FITC,

polyA of 38 bp: 5′-NH₂—(CH₂)₁₂—PO₄-A₃-3′-FITC,

Single-stranded random sequence of 19 bp: 5′-NH₂—(CH₂)₁₂—PO₄-TGGGCTGGCCAAACTGCTG-3′-FITC, (Seq ID No. 1) and

double-stranded random sequence of 19 bp: (Seq ID No. 2 and Seq ID No. 3)

```
                                3′-ACCCGACCGGTTTGACGAC-5′
                                      |||||||||||||||||||
5′-NH2-(CH2)12-PO4-TGGGCTGGCCAAACTGCTG-3′-FITC
```

all of which are synthesized by invitrogen Trading Shanghai. 2. Cell Preparation Before Transfer of Single-Stranded or Double-Stranded DNA/RNA in Different Sequences

24 h before transfer, the HepG2 cells in the phase of logarithmic growth were digested with trypsin; a culture medium containing 10% serum was used for adjusting the cell density to 0.5×10⁶ cells/mL; and the cells were inoculated again in a cell culture dish of 15 cm and cultured in a culture incubator containing 5% CO₂ at 37° C. The cells may be used for experiments when the cell density reaches 60% to 70% 24 h later.

3. Transfer of Single-Stranded or Double-Stranded DNA/RNA

4 nmol of synthesized single-stranded or double-stranded DNA/RNA fragments in different sequences was added to a sterile centrifuge tube (tube A) of 15 mL, respectively, and uniformly mixed with Opti-MEM in a corresponding volume, with a total volume of 2 mL; the X-tremeGENEsiRNA reagent was shaken gently, and 160 μL of X-tremeGENEsiRNA reagent was mixed with 1.84 mL of Opti-MEM in another tube (tube B); and the solution in the tube A was mixed with the solution in the tube B, the mixture was slightly triturated by a pipette and incubated for 20 min at room temperature.

6 mL of RPMI-1640 serum-free culture medium was added to the mixture and mixed uniformly; the primary culture medium in the HepG2 cell culture dish was discarded, and slightly triturated with RPMI-1640 serum-free culture medium once; and then, the mixture was moved into the HepG2-PT cell culture dish, and cultured in a culture incubator containing 5% CO₂ at 37° C. 6 h later, the positioning of the DNA/RNA in the cells was observed by a laser confocal microscope.

4. Experimental Results

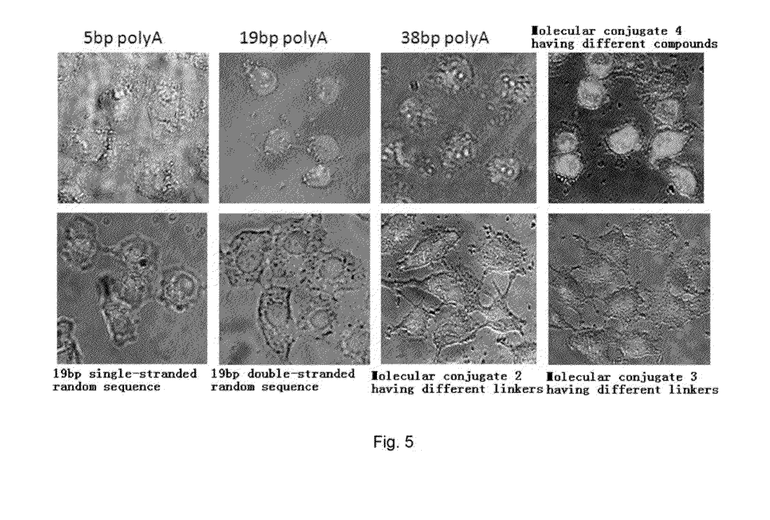

The results are as shown in FIG. 5, polyA of 5 bp, polyA of 19 bp, polyA of 38 bp, single-stranded random sequence fragments of 19 bp and double-stranded random sequence fragments of 19 bp may be all transferred into cells by X-tremesiRNA, with most of them into the cytoplasm and a few of them into the cell nucleus.

### Embodiment 3

### Evaluation on Transmembrane Transfer Efficiency of Molecular Conjugates

1. Materials and Reagents

The HepG2 cell strains were purchased from Shanghai Institutes for Bioscience Chinese Academy of Sciences; the RPMI-1640 culture medium was purchased from Hyclone Shanghai; the fetal bovine serum was purchased from Tianjin Hao Yang Biological Products Co., Ltd.; the trypsin and Opti-MEM were purchased from Invitrogen Shanghai; the X-tremeGENEsiRNA transfection reagent was purchased from Roche China; and the cell culture dishes and other consumables were all purchased from Corning China. 2. Cell preparation before transfer of the molecular conjugates

24 h before transfer, the HepG2 cells in the phase of logarithmic growth were digested with trypsin; a culture medium containing 10% serum was used for adjusting the cell density to 0.5×10⁶ cells/mL; and the cells were inoculated again in a cell culture dish of 15 cm and cultured in a culture incubator containing 5% CO₂ at 37° C. The cells may be used for experiments when the cell density reaches 60% to 70% 24 h later.

3. Transfer of the Molecular Conjugates

4 nmol of molecular conjugates 1, 2, 3, 4 (prepared by the synthesis routes of Embodiment 1) and FITC-separately-tagged 4-bromo-3-oxo-ethyl cyclopropanecarboxylate-5-(3-((1-phenyldiethyl carbamoylpiperidine)-4-methyl)phenyl)thiophene-2-methyl formate were add to five sterile centrifuge tubes (labeled as tubes A1, A2, A3, A4 and A5, respectively) of 15 mL, and uniformly mixed with Opti-MEM in a corresponding volume, with a total volume of 2 mL.

1.84 mL of Opti-MEM was added to additional five sterile centrifuge tubes (labeled as tubes B1, B2, B3, B4 and B5, respectively) of 15 mL; and the X-tremeGENEsiRNA reagent was shaken gently, and 160 μL of X-tremeGENEsiRNA reagent was added to tubes B1, B2, B3, B4 and B5, respectively and mixed uniformly.

The solutions in tube A and tube B having a corresponding number, for example, A1 and B1, were mixed, slightly triturated by a pipette, and incubated for 20 min at room temperature.

6 mL of RPMI-1640 serum-free culture medium was added to the mixture and mixed uniformly; the primary culture medium in the HepG2 cell culture dish was discarded, and slightly triturated with RPMI-1640 serum-free culture medium once; and then, the mixture was moved into a HepG2-PT cell culture dish, and cultured in a culture incubator containing 5% CO₂ at 37° C. After 6 hours, the situation of positioning of the molecular conjugate 1, 2, 3, 4 in cells is observed by using a laser confocal microscope.

4. Experimental Results

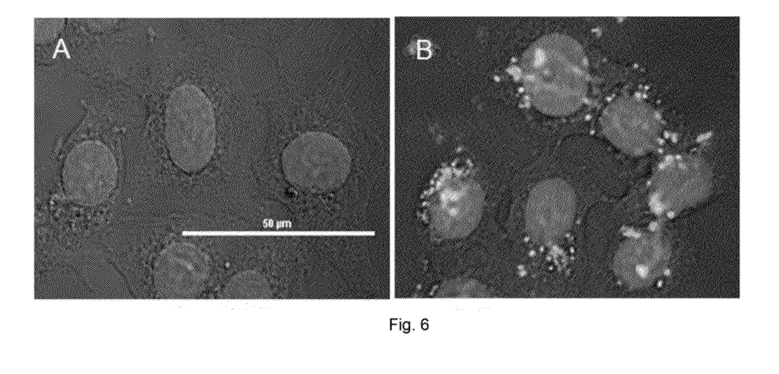

As shown in FIG. 5 and FIG. 6, the molecular conjugates 1, 2, 3, 4 may penetrate through the cell membrane successfully and be transferred into cells.

(2) As shown in FIG. 6A, 4-bromo-3-oxo-tert-Butyl acetate-5-(3-((((1-phenyl carbamoylpiperidine)-4-methyl)-N-propargyl amine)phenyl)thiophene-2-methyl formate directly tagged with FITC cannot penetrate through the cell membrane into cells; as shown in FIG. 6B, the molecular conjugates 1 linked with the DNA/RNA on the basis of an individual compound may be transferred into cells, with most of them into the cytoplasm and a few of them into the cell nucleus.

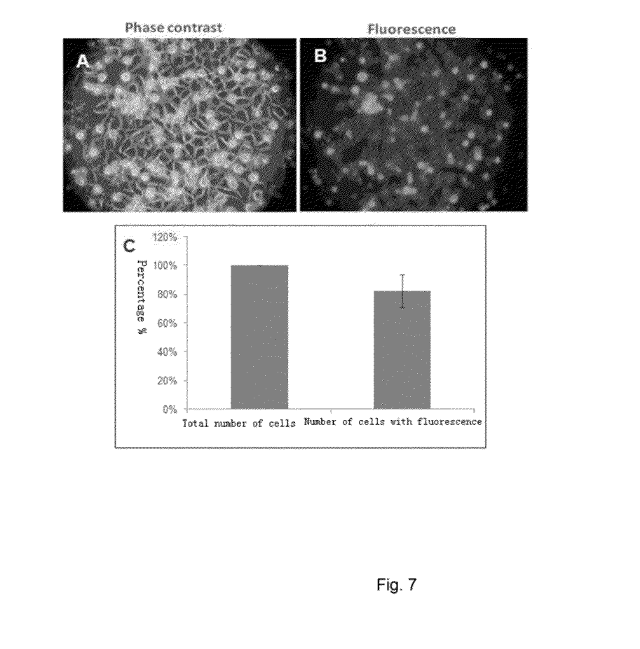

(3) The transfer efficiency was recorded by observation by a microscope: FIG. 7A shows the total number of cells in transfer experiments, observed by a phase contrast microscope; FIG. 7B shows the total number of cells into which the molecular conjugates 1 are successively transferred, observed by a fluorescent microscope; and as shown in FIG. 7C, it may be known from statistical results and calculation that the transfer efficiency of the molecular conjugates 1 may achieve over 80%.

### Embodiment 4

### Study on Influence of Molecular Conjugates on Membrane Transfer of Compounds

1. Experimental Materials and Reagents

The HepG2 cell strains were purchased from Shanghai Institutes for Bioscience Chinese Academy of Sciences; the RPMI-1640 culture medium was purchased from Hyclone Shanghai; the fetal bovine serum was purchased from Tianjin Hao Yang Biological Products Co., Ltd.; the trypsin was purchased from Invitrogen; the cell lysis buffer and the protease inhibitor were purchased from Pierce; the P-IRS-1 ELSA kit was purchased from Bio-swamp; and the cell culture dishes and other consumables were all purchased from Corning.

2. Study on Influence of Molecular Conjugates on Membrane Transfer of Compounds

Protein tyrosine phosphatase-1B (PTP1B), belonging to the family of protein tyrosine phosphatases (PTPs) and existing in two forms of transmembrane receptor-like protein and endoenzyme, catalyzes the dephosphorylation reaction of phosphorylated tyrosine residues of protein, and is the first PTPs identified and purified in mammalian bodies. PTP1B acts on proteins related to insulin-signaling transduction, such as, insulin receptor (IR), insulin receptor substrates 1, 2 (IRS-1, IRS-2), growth factor receptor bound protein 2 (Grb2) and phosphatidylinositol 3 kinase (PI-3K), so that the phosphorylated tyrosine residues of these proteins are dephosphorylated, thereby attenuating the insulin-signaling transduction, thus producing post-receptor insulin resistance. Since it is known that the raw material compound 4-bromo-3-oxo-tert-butyl acetate-5-(3-(((1-phenyl carbamoylpiperidine)-4-methyl)-N-propargyl amine)phenyl)thiophene-2-methyl formate plays a same role as PTP1B inhibitor (J. Med. Chem. 2007, 50, 4681-4698), in the present invention, by measuring change in IRS-1 phosphorylation level when the molecular conjugates 1 are transferred into cells, it is determined that the compound indeed goes into cells in the form of molecular conjugates after being covalently linked to the DNA/RNA, and it is possible to effectively affect the function of the insulin-signaling pathway. A verification method is as follows:

(1) 24 h before transfer, the HepG2 cells in the phase of logarithmic growth were digested with trypsin; a culture medium containing 10% serum was used for adjusting the cell density to 0.5×10⁶ cells/mL; and the cells were inoculated again in a six-pore plate and cultured in a culture incubator containing 5% CO₂ for 24 h at 37° C. The cells may be used for experiments when the cell density achieves 60% to 70% 24 h later.

(2) 0.025 μg of molecular conjugates 1 and 0.075 μg of molecular conjugates 1 were added to two sterile centrifuge tubes (tubes C1, C2) of 1.5 mL, respectively, and uniformly mixed with Opti-MEM in a corresponding volume, with a total volume of 100 μL; the X-tremeGENEsiRNA reagent was shaken gently, and 2.5 μL of X-tremeGENEsiRNA reagent was mixed with 97.5 μL of Opti-MEM in other two tubes (tubes D1, D2), with a total volume of 100 μL; and the solution in the tube C was mixed with the solution in the tube D, for example, C1 and D1, slightly triturated by a pipette, and incubated for 20 min at room temperature. Operations similar to the above were repeated, with cases without compounds or without X-tremeGENEsiRNA or without both as two contrast controls and a blank control, respectively.

(3) 800 μL of RPMI-1640 serum-free culture medium was added to the mixture and mixed uniformly; the primary culture medium in the HepG2 cell culture dish was discarded, and slightly washed with RPMI-1640 serum-free culture medium once; then the mixture obtained in the step (2) was moved into the HepG2 cell culture dish, and cultured in a culture incubator containing 5% CO₂ at 37° C. for 5 h, the case without the X-tremeGENEsiRNA transfection reagent and the molecular conjugates 1 as blank control. 1 μg/mL of insulin and glucose (5 mM) were added for induction for half an hour.

(4) The cells were washed with ice-cold PBS for three times; 50 μL of cell lysis buffer was added in each pore for lysis on ice for 1 h; and then, the solution was centrifuged, the supernatant was collected, and protein quantization was performed by a BCA kit. A same amount of total protein was added into an ELISA plate, and the phosphorylation level was measured by an ELISA kit. Four parallel pores were designed in each experiment, and the data came from three independent experiments.

3. Experimental Results

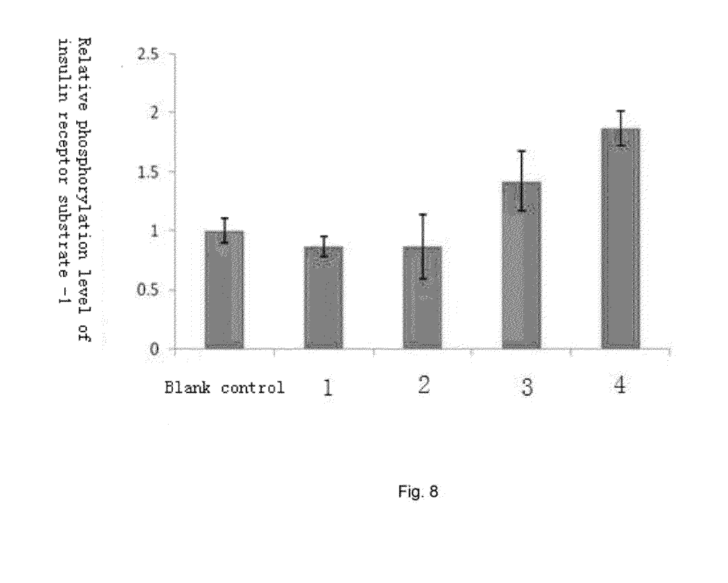

As shown in FIG. 8, after the molecular conjugates having different concentration were transferred into HepG₂ cells, compared with the blank control without molecular conjugates, the phosphorylation level of IRS-1 in the cells is increased and positively corresponds to the concentration of the compounds. It is shown that the compounds indeed go into the cells together with the DNA/RNA after being covalently linked to the DNA/RNA, and may play their original functions in the form of molecular conjugates.

In conclusion, the cell-penetrating method of the present invention may effectively transfer compounds with low membrane permeability into cells. The cell-penetrating method is easy in operation and high in transfer efficiency, and can maintain the activity of the compounds to the maximum extent, and is safe and non-poisonous. The cell-penetrating method provides a new approach to clinical treatment of drugs with low membrane permeability. This method significantly increases the quantity of potential drugs, and the clinical application of many drugs which are eliminated due to their low membrane permeability becomes possible. Furthermore, the method of the present invention may be used for capturing unknown targets of drugs in cells and conducting researches on the target mechanism. This method significantly shortens the course of research and development of drugs and has excellent application prospect.

We claim:

1. A cell-penetrating method for compounds, comprising the following steps of:
(1) Preparing raw materials, i.e., the compounds and DNA or RNA;
(2) Linking: linking the compounds to the DNA or RNA to obtain a molecular conjugate; and
(3) Transferring: transferring the molecular conjugate obtained in the step (2) into cells by a gene transfer method.

2. The method according to claim 1, characterized in that, in the step (1), the compounds are small-molecule compounds or polypeptides having a molecular weight of 100 Da to 4000 Da.

3. The method according to claim 1, characterized in that, in the step (1), the DNA or RNA is any sequence having a length not less than five bases or base pairs.

4. The method according to claim 1, characterized in that, in the step (1), the DNA or RNA is single-stranded or double-stranded.

5. The method according to claim 4, characterized in that an end-chain or in-chain covalent bond is bound with zero or a plurality of tags.

6. The method according to claim 5, characterized in that the tag is fluorescent or isotopic.

7. The method according to claim 1, characterized in that, in the step (2), the compounds are linked to the DNA or RNA by a linker.

8. The method according to claim 7, characterized in that the linker is formed by linking any saturated and non-saturated covalent groups capable of modifying the compounds and the DNA or RNA.

9. The method according to claim 1, characterized in that, in the step (3), the gene transfer method refers to a cationic liposome transfection method, a calcium phosphate transfection method, a nanoparticles transfection method or an electroporation transfection method and other technical methods capable of transferring nucleic acid into cells.

10. A molecular conjugate, having a structural formula as follows:
X-linker-DNA/RNA Formula 1
where X refers to compounds with low membrane permeability, and linker is a linker between X and DNA or RNA.

11. The molecular conjugate according to claim 10, characterized in that, in the step (1), the compounds are small-molecule compounds or polypeptides having a molecular weight of 100 Da to 4000 Da.

12. The molecular conjugate according to claim 10, characterized in that the DNA or RNA is any sequence having a length not less than five bases or base pairs.

13. The molecular conjugate according to claim 10, characterized in that the DNA or RNA is single-stranded or double-stranded.

14. The molecular conjugate according to claim 13, characterized in that an end-chain or in-chain covalent bond is bound with zero or a plurality of tags.

15. The molecular conjugate according to claim 14, characterized in that the tag is fluorescent or isotopic.

16. The molecular conjugate according to claim 10, characterized in that the linker is formed by linking any saturated and non-saturated covalent groups capable of modifying the compounds and the DNA or RNA.


SEQUENCE LISTING (3 sequences)

SEQ ID NO: 1 - DNA, 19 bp, Artificial Sequence
  synthetic
tgggctggccaaactgctg

SEQ ID NO: 2 - DNA, 19 bp, Artificial Sequence
  synthetic
acccgaccggtttgacgac

SEQ ID NO: 3 - DNA, 19 bp, Artificial Sequence
  synthetic
tgggctggccaaactgctg